(19) United States
(12) Patent Application Publication
(10) Pub. No.: US 2010/0261949 A1
(43) Pub. Date: Oct. 14, 2010

(54) Method of Training a Living Body to not React to Substances

(76) Inventor: Dawn Nowlin, Leesburg, VA (US)

Correspondence Address: GREENBERG & LIEBERMAN, LLC, 2141 WISCONSIN AVE, N.W., SUITE C-2, WASHINGTON, DC, 20007, US

(21) Appl. No.: 12/679,450

(22) PCT Filed: Sep. 22, 2008
(86) PCT No.: PCT/US08/77237

§ 371 Date: Jun. 18, 2010

Related U.S. Application Data
(60) Provisional application No. 60/974,195, filed on Sep. 21, 2007.

(51) Int. Cl. A61M 21/00

(52) U.S. Cl. 600/28

(57) ABSTRACT

A method for training a living body of a patient to attribute sounds corresponded with allergens to ultimately cease negative reactions to such allergens or substances. In addition, a patient's allergies are identified by using sound as opposed to exposing the patient to the actual substances. Acupressure is used in conjunction with the sounds to train the body to react positively to the allergens without actual allergen substance being exposed to the patient.

This is a non-provisional application claiming priority to provisional patent application No. 60/974,195 filed on Sep. 21, 2007.

### FIELD OF THE INVENTION

The present invention is a method of training a living body of a patient to attribute sounds corresponded with allergens to ultimately cease negative reactions to such allergens or substances. In addition, the method of the present invention serves to identify a patient's allergies by using sound as opposed to exposing the patient to the actual substances.

### BACKGROUND OF THE PRESENT INVENTION

Allergies and sensitivities are the result of the immune system reacting inappropriately to harmless, naturally occurring substances, which can affect virtually every part of the body. This bodily reaction is abnormal. The effects of allergies can range from mild symptoms to extremely harmful reactions. Sensitivities are another abnormal set of reactions. Although they have symptoms similar to those of allergies, sensitivities do not involve the immune system.

It has been observed that exposure to minute amounts of an allergic or reactive substance to the surface of the skin causes a body's muscles to immediately become weakened due to contact with the specific substance. Although no other reactions may occur from this minimal, external exposure, it has been observed that weakened muscle strength is a universal physiological response to exposure to any allergic or reactive substance. The inventor concluded that the body is recognizing the substance as “information” and the response depends on the interpretation of the information. If the body perceives the substance as harmful, it will cause an immediate physiological response. If the body perceives the substance as benign, no physiological change occurs.

The present invention takes advantage of this phenomenon, and takes it to the next degree. According to the science of biosemiotics and information theory, signals or information can be read at the molecular level in all living systems. Human physiology operates entirely on signals of information. In the case of allergies and sensitivities, the body is in error, or is incorrect in its perception of a substance. By exposing the person to the substance while simultaneously introducing a positive stimulus, the body associates the positive stimulus with the benign substance and alters its perception, interpreting the substance as beneficial.

The inventor has found that if a digital sample of the substance is transmitted to the surface of the skin, the body responds in a similar or identical fashion as if they were exposed to the actual substance. It has also been found that this bodily reaction is controlled at the subconscious level, and as such, the sound does not have to be within the range of normal human hearing capacity. The digital signal is initially created from a textual representation, including a specific sequence of words that allows for a more specific representation of the substance. This textual representation is converted to an audio signal, which has then been converted to a specific tone that represents the substance. The inaudible tone is transmitted to surface of the skin through a wrist cuff.

### SUMMARY OF THE PRESENT INVENTION

The method of the present invention is as follows: While an inaudible sound, which represents the substance the body is inappropriately reacting to, is transmitted to the body, acupuncture points are pressed evenly down both sides of the spine on the Bladder Meridian, as known in Traditional Chinese Medicine. These points are utilized to temporarily strengthen all of the major organ systems. The combination of the subconscious bodily reaction to the inaudible sound and the acupressure along the spine teaches the body to not react to that particular substance or “information”. The concept that the body as a whole, not just the immune system, perceives an allergen as a threat and overreacts is the basis of the present invention.

It is the same phenomenon with sensitivities; only allergies must involve the immune system to be officially called allergies. This association causes the body to perceive the substance as a benefit, instead of as a threat. It is not unlike allergy shots, where the goal is to slowly train the body to accept highly diluted amounts of the allergen. Over time, the body is trained to accept the substance. The present invention introduces the exposure along with the positive stimulus, which allows a much faster process of accepting the substance and a resolution to the allergic reactions.

The therapeutic stimulation used in conjunction with the transmission of digital signals is the mechanism of action in training the body to accept a harmless substance. The transmission of digital signals has no effect or therapeutic value to the body. It is only the acupressure stimulation in conjunction with exposure to the digital representation that allows for a resolve in the physiological error.

The inventor has discovered that the success of the therapy depends on the accuracy of the information transmitted to the body. Therefore, a database is used to include a large number of substances and further breaks the substances down into components and further into components of the components down to the actual protein molecule. This methodology allows for superior precision in biological communication.

Provided herein as part of this application is a compendium of many of the substances and some of the elements thereof, to which people can react as allergens, the software which physicians may use in order to find that particular allergen, and a schematic for the cuff, which wraps around the patient's arm and is connected to the computer system which transmits the inaudible sounds.

### BRIEF DESCRIPTION OF THE DRAWINGS

FIG. 1 is a flow chart detailing the steps of the preferred embodiment of the present invention.

### DETAILED DESCRIPTION OF THE PREFERRED EMBODIMENT

The present invention is a method that serves to find inaudible sounds associated with a specific allergen and/or sensitivity in order to ultimately train the body's natural physiology to accept such specific protein or irritant that initially caused the allergic or sensitivity reaction. For example, a database of the present invention may house the inaudible sounds associated with cat hair. The patient would hold his or her arm in a certain way as various inaudible sounds associated with cat hair allergies are transmitted to the surface of the skin The patient will not be able to maintain the arm position and the muscle will weaken in reaction to the negative effect of the inaudible sound representative of the specific allergen. In this manner, it will become known through the database which overall allergen is the problem, as well as which inaudible sound associated with such an allergen or irritant. So, in this example, cat hair can be singled out as an irritant or allergen.

Once the negative influence on the body is determined based on the muscle response, different inaudible sounds are projected to hone in on the more specific cause of the irritation. Each of these different inaudible sounds is associated with different proteins or other sub-levels of the allergen. In the cat hair example, this would mean individual proteins within the cat hair will be probed via the different inaudible sounds. Such proteins include items as saliva, dander, etc. In other words, the different inaudible sounds seek muscular reaction to determine which part of the broader-known allergen or irritant is affecting the patient. The determination will be made when the muscle reacts to the targeted projection of the different inaudible sound.

Acupressure is then applied to certain points on the body. In the preferred embodiment, acupuncture points are pressed evenly down both sides of the spine on the Bladder Meridian. These points are utilized to temporarily strengthen all of the major organ systems. Meanwhile, the different inaudible sound specific to the distinct substance found to cause the irritation is transmitted onto the body. The combination of the subconscious bodily reaction to the inaudible sound and the acupressure along the spine teaches the body to not react to that particular substance or “information”. In other words, the different inaudible sound that represents an allergen or irritant transmitted at the same time as stimulating positive pressure points on the body ultimately trains the body to associate the allergen or irritant with positive benefit of the stimulation.

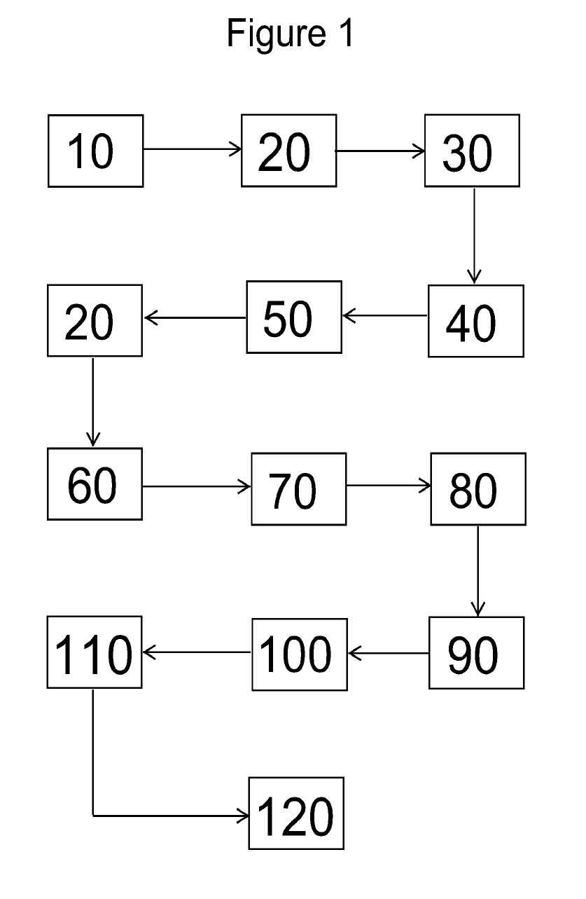

As we see in FIG. 1, the present invention can proceed as follows. First, a speaker is positioned in the proximity of the patient (10). It should be noted that the patient also might be referred to as person. In one embodiment, the speaker may be placed on the underside of a wristband. The wristband would then be wrapped around the wrist, leg, or otherwise around an extremity of the patient. The speaker would be facing the body of the patient. Then, a sound is transmitted toward the body of the patient (20). The sound being transmitted is representative of what the actual potential allergen or reactive substance is purported to be. This means that the sound literally can be a grouping of words identifying the purported allergen. The sound being transmitted, however, will be unintelligible to the patient. This is because in the preferred embodiment, the sound is converted into a digital format (30) so as to be emitted in a digital format (40). An additional embodiment permits the sound to be encrypted (50) via conventional means. Regardless, the sound being transmitted comes out unintelligible to the patient as the conventional representative words are either inaudibly below or above the frequency for hearing ability of a human, or garbled via digital and encryption avenues. While the sound is transmitted toward the body of the patient (20), the muscle strength of the patient is monitored (60). The monitoring of the muscle strength also includes testing such as resistance force. The muscle strength of the patient will remain optimal while sounds are transmitted. However, once a sound representing an allergen or irritant prevalent with the patient reaches the body of the patient (70), the patient's muscle strength will noticeably decrease (80). The loss of muscle strength will be recorded (90) in conjunction with the certain sound. That certain sound will identify the particular allergen that caused the loss of muscle strength (100). Once the sound representing a particularly relevant allergen or irritant is identified and matched, the sound is again transmitted toward the body of the patient (110) in much the same manner as the previous times. But this time, pressure points on the patient's body via conventional acupressure are pressed while the sound representing the identified allergen is transmitted (120). This has the effect of teaching the patient's body to attribute the allergen or irritation as a physical positive, which in turn will diminish the veracity of the allergen's effect on the patient's body.

The preferred embodiment of the present invention is a method of training a living body of a patient, comprising positioning a speaker in the proximity of the patient, transmitting a series of sounds from the speaker toward the body of the patient, each sound of the series of sounds matched with a corresponding allergen, monitoring and testing muscle strength of the patient, and recording the corresponding allergen matched with each sound of the series of sounds that causes muscle strength to decrease. In addition, the present invention entails transmitting toward the body of the patient each sound of the series of sounds that causes muscle strength to decrease and pressing pressure points via acupressure while each sound of the series of sounds that causes muscle strength to decrease is further transmitted.

Moreover, facing the speaker toward the body of the patient is part of the preferred embodiment, along with converting each sound of the series of sounds into a digital format and encrypting each sound of the series of sounds. Storing and matching each sound of the series of sounds with the corresponding allergen in a database also is envisioned. An alternative embodiment involves placing the speaker onto a band, the band configured to secure to an extremity of the patient. Each sound of the series of sounds also may be converted into an unintelligible format.

I claim:

1. A method of training a living body of a patient, comprising:
positioning a speaker in the proximity of the patient;
transmitting a series of sounds from the speaker toward the body of the patient, each sound of the series of sounds matched with a corresponding allergen;
monitoring and testing muscle strength of the patient; and
recording the corresponding allergen matched with each sound of the series of sounds that causes muscle strength to decrease.

2. The method of claim 1, further comprising further transmitting toward the body of the patient each sound of the series of sounds that causes muscle strength to decrease.

3. The method of claim 2, further comprising pressing pressure points while each sound of the series of sounds that causes muscle strength to decrease is further transmitted.

4. The method of claim 1, further comprising facing the speaker toward the body of the patient.

5. The method of claim 1, further comprising converting each sound of the series of sounds into a digital format.

6. The method of claim 1, further comprising encrypting each sound of the series of sounds.

7. The method of claim 1, further comprising storing and matching each sound of the series of sounds with the corresponding allergen in a database.

8. The method of claim 1, further comprising placing the speaker onto a band, the band configured to secure to an extremity of the patient.

9. The method of claim 1, further comprising converting each sound of the series of sounds into an unintelligible format.

10. The method of claim 3, further comprising pressing pressure points via acupressure while each sound of the series of sounds that causes muscle strength to decrease is further transmitted.

11. The method of claim 2, further comprising facing the speaker toward the body of the patient.

12. The method of claim 2, further comprising converting each sound of the series of sounds into a digital format.

13. The method of claim 2, further comprising encrypting each sound of the series of sounds.

14. The method of claim 2, further comprising storing and matching each sound of the series of sounds with the corresponding allergen in a database.

15. The method of claim 2, further comprising placing the speaker onto a band, the band configured to secure to an extremity of the patient.

16. The method of claim 2, further comprising converting each sound of the series of sounds into an unintelligible format.

17. A method of training a living body of a patient, comprising:
positioning a speaker in the proximity of the patient;
transmitting a sound from the speaker toward the body of the patient, the sound matched with a corresponding allergen; and
pressing pressure points via acupressure while transmitting the sound from the speaker.

18. A method of training a living body of a patient, comprising:
positioning a speaker in the proximity of the patient;
transmitting a series of sounds from the speaker toward the body of the patient, each sound of the series of sounds matched with a corresponding allergen;
monitoring and testing muscle strength of the patient;
recording the corresponding allergen matched with each sound of the series of sounds that causes muscle strength to decrease;
further transmitting toward the body of the patient each sound of the series of sounds that causes muscle strength to decrease;
pressing pressure points via acupressure while each sound of the series of sounds that causes muscle strength to decrease is further transmitted;
facing the speaker toward the body of the patient;
converting each sound of the series of sounds into a digital format;
encrypting each sound of the series of sounds;
storing and matching each sound of the series of sounds with the corresponding allergen in a database;
placing the speaker onto a band, the band configured to secure to an extremity of the patient; and
converting each sound of the series of sounds into an unintelligible format.
